(19) United States
(12) Patent Application Publication
(10) Pub. No.: US 2023/0248952 A1
(43) Pub. Date: Aug. 10, 2023

(54) METHODS FOR DELIVERING BIOACTIVE COMPOSITIONS AND FORMULATIONS TO THE SKIN USING MICROCHANNEL DELIVERY DEVICE

(71) Applicant: AQUAVIT PHARMACEUTICALS, INC., Las Vegas, NV (US)

(72) Inventors: Sobin Chang, Las Vegas, NV (US); Saiprasad Sankaranarayan, Las Vegas, NV (US)

(21) Appl. No.: 18/008,037

(22) PCT Filed: Jun. 4, 2021
(86) PCT No.: PCT/US21/35978

§ 371 Date: Dec. 2, 2022

Related U.S. Application Data
(60) Provisional application No. 63/034,906, filed on Jun. 4, 2020.
(60) Provisional application No. 63/079,328, filed on Sep. 16, 2020.

(51) Int. Cl. A61M 37/00

(52) U.S. Cl. CPC A61M 37/0015; A61M 2037/0023; A61M 2037/0061; A61M 2037/0038

(57) ABSTRACT

The present invention provides a method for treating a condition or disease in a subject, comprising administering to the subject's skin a composition comprising an effective amount of one or more bioactive formulations and compositions, including neurotoxins, wherein the composition is administered with a single-chamber or multi-chamber microchannel delivery device.

This application claims the benefit of the U.S. Provisional Appl. No. 63/034,906, filed on Jun. 4, 2020, and U.S. Provisional Appl. No. 63/079328, filed on Sep. 16, 2020, the contents of which are hereby incorporated by reference in their entirety.

### FIELD OF THE INVENTION

The field of the invention relates generally to the field of medicine, specifically designs and methods used for treating diseases or conditions, e.g., dermatologically, psychologically, by delivering one or more bioactive compositions such as vitamins, hyaluronic acid, neurotoxins, cannabinoids, vaccines, etc.

### SUMMARY OF THE INVENTION

It is to be understood that both the foregoing general description of the embodiments and the following detailed description are exemplary, and thus do not restrict the scope of the embodiments.

In one aspect, the invention provides a method for treating a disease or condition in a subject, administering to the subject's skin a composition comprising an effective amount of one or more neurotoxins, wherein the composition is administered with a single-chamber or multi-chamber microchannel delivery device.

In another aspect, the invention provides a microchannel delivery device comprising an effective amount of one or more neurotoxins for use in the methods herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1 is an exploded 3D drawing of the internal part of a microchannel delivery device. The diagram shows the reservoir that holds the composition including neurotoxins. The reservoir is connected to the microneedle head by a neck. The neck contains grooves that enable the microneedle head to be attached to the reservoir. The microneedle head contains a set of microneedles that enable delivery of composition from the reservoir to the administered region. The microneedle head consists of the following components: microneedles and housing of the needles. The microinjection of composition is functioned via a plurality of microneedles.

FIG. 2 illustrates an assembled internal part of the microchannel drug delivery device.

FIG. 3 illustrates an assembled internal part of the microchannel drug delivery device.

FIG. 4 illustrates an exploded 3D drawings of the external push assembly of a microchannel delivery device. The diagram illustrates a cap, a base assembly part and spring part. The base assembly holds the internal part of the microchannel delivery device. The cap closes the base assembly and encloses the internal part of the microchannel delivery device. The top portion of the cap is open to enable the microneedle head to be exposed. The spring enables the push and tap mechanism of the device. The push and tap mechanism enables the internal part to push towards the cap, enabling the microneedle head to be exposed thereby assisting in administration of composition from the reservoir through the microneedle head.

FIG. 5 illustrates an assembled microchannel drug delivery device containing internal parts and external push assembly components.

FIG. 6 illustrates an assembled microchannel drug delivery device containing internal parts and external push assembly components.

FIG. 7 illustrates a multi chamber microneedle drug delivery device design that features a pusher that is activated by the subject. The pusher pierces the layer separating Chamber I and Chamber II thereby allowing flow of bioactive composition from chamber I to chamber II. After this, the bioactive compositions are mixed or reconstituted by one of the following methods: gravity-driven motion, pressure infused motion, electrically powered systems. After this the bioactive composition transfers to the reservoir, and can be administered on a subject. The microchannel head facilitates movement from the reservoir to the subject's skin.

FIG. 8 illustrates a multi chamber microneedle drug delivery device design that features a pusher that is activated by the subject. The pusher pierces the layer separating Chamber I and Chamber II thereby allowing flow of bioactive composition from chamber I to chamber II. After this, the bioactive compositions are mixed or reconstituted by one of the following methods: gravity-driven motion, pressure infused motion, electrically powered systems. After this the bioactive composition transfers to the reservoir, and can be administered on a subject. The microchannel head facilitates movement from the reservoir to the subject's skin.

FIG. 9 illustrates a modular multi chamber microneedle drug delivery device design. This allows the chambers and the reservoir with the microneedle head to be detachable. The chambers can be replaced or substituted. It features a pusher that is activated by the subject. The pusher pierces the layer separating Chamber I and Chamber II thereby allowing flow of bioactive composition from chamber I to chamber II. After this, the bioactive compositions are mixed or reconstituted by one of the following methods: gravity-driven motion, pressure infused motion, electrically powered systems. After this the bioactive composition transfers to the reservoir, and can be administered on a subject. The microchannel head facilitates movement from the reservoir to the subject's skin.

FIG. 10 illustrates a multi chamber microneedle drug delivery device design that features a pusher that is activated by the subject. The pusher pierces the layer separating Chamber I and Chamber II thereby allowing flow of bioactive composition from chamber I to chamber II. After this, the bioactive compositions are mixed or reconstituted by one of the following methods: gravity-driven motion, pressure infused motion, electrically powered systems. After this the bioactive composition transfers to the reservoir, and can be administered on a subject. The microchannel head facilitates movement from the reservoir to the subject's skin. It also features a blender that can be activated by the subject through an external button/switch. This blender helps in auto-reconstitution of the bioactive formulations in the chambers.

FIG. 11 illustrates a multi chamber microneedle drug delivery device design that features multiple pushers that are activated individually or together by the subject. Each pusher pierces the layer separating the two chambers thereby allowing flow of bioactive composition from one chamber to another. After this, the bioactive compositions are mixed or reconstituted by one of the following methods: gravity-driven motion, pressure infused motion, electrically powered systems. After this the bioactive composition transfers to the reservoir, and can be administered on a subject. The microchannel head facilitates movement from the reservoir to the subject's skin. Each of these chambers can contain different compositions.

FIG. 12 illustrates a modular multi chamber microneedle drug delivery device design that features multiple chambers that can be attached to each other. Each chamber features a pusher that pierces the layer separating the two chambers thereby allowing flow of bioactive composition from one chamber to another. After this, the bioactive compositions are mixed or reconstituted by one of the following methods: gravity-driven motion, pressure infused motion, electrically powered systems. After this the bioactive composition transfers to the reservoir, and can be administered on a subject. The microchannel head facilitates movement from the reservoir to the subject's skin. Each of these chambers can contain different compositions.

FIG. 13 illustrates a modular multi chamber microneedle drug delivery device design that features two chambers that can be attached to each other wherein one chamber contains the pusher that pierces the other chamber. The pusher pierces the outer layer of the attached chamber thereby allowing flow of bioactive composition from one chamber to another. After this, the bioactive compositions are mixed or reconstituted by one of the following methods: gravity-driven motion, pressure infused motion, electrically powered systems. After this the bioactive composition transfers to the reservoir, and can be administered on a subject. The microchannel head facilitates movement from the reservoir to the subject's skin. Each of these chambers can contain different compositions.

FIG. 14 illustrates a microchannel head adapter that fits regular hypodermic syringes. This facilitates the bioactive composition to flow from regular syringes to the site of administration through the microneedles

FIG. 15 illustrates an exemplary assembled microchannel drug delivery device containing a syringe plunger, a reservoir and a microneedle head. The plunger movement facilitates active flow of bioactive composition from the reservoir to the microneedle head, to be administered on a subject.

FIG. 16 illustrates the method of utilizing the microchannel drug delivery device to load and administer composition.

### DETAILED DESCRIPTION OF THE INVENTION

Reference will now be made in detail to the presently preferred embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skills in the art.

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3rd ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the term “about” means plus or minus 10% of the numerical value of the number with which it is being used.

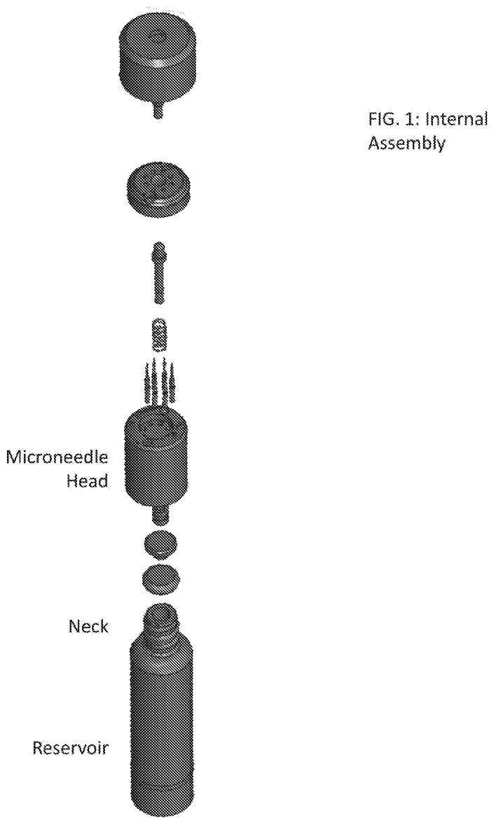

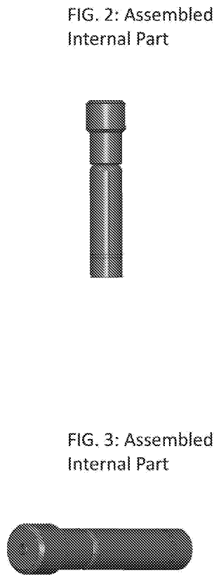

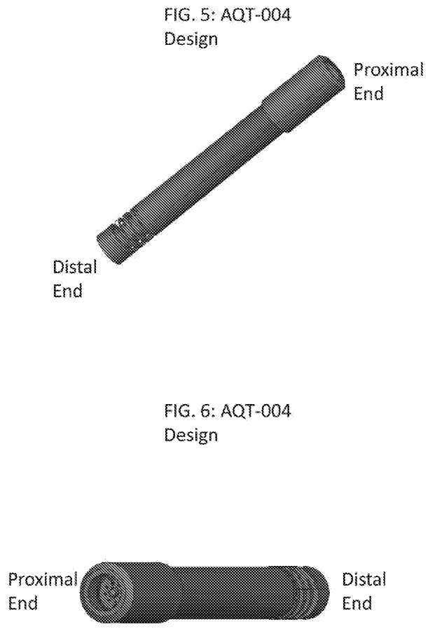

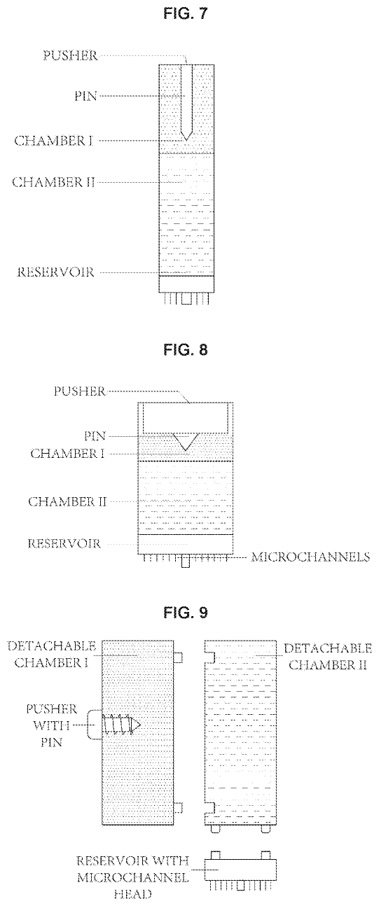

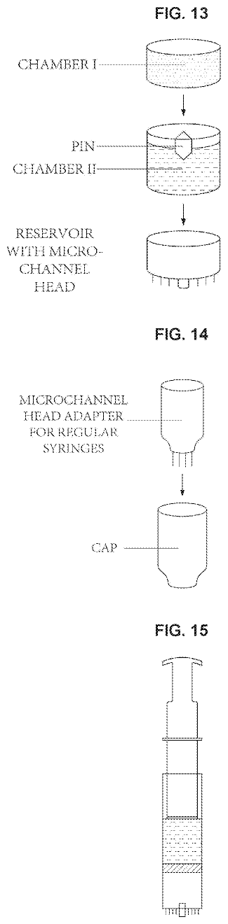

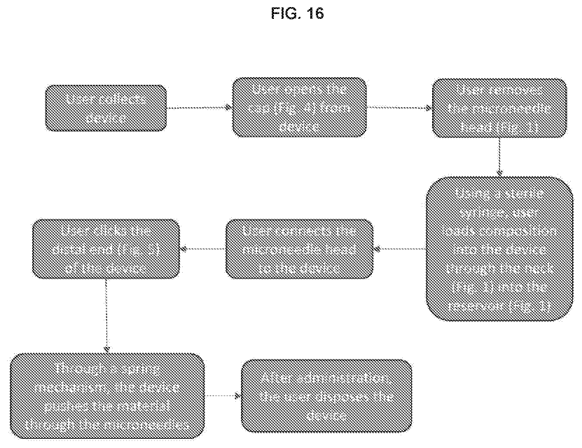

In one embodiment, the invention provides a method for treating a disease or condition in a subject, administering to the subject's skin a composition comprising an effective amount of one or more neurotoxins, wherein the composition is administered with a microchannel delivery device. In some embodiments, the microchannel delivery device useful in the methods of the invention is depicted in FIGS. 1-15.

In some embodiments, the treatment methods further comprise administering to the subject one or more additional therapies. In some embodiments, the additional therapy can include one or more therapies selected from radiation, surgery, chemotherapy, simple excision, Mohs micrographic surgery, curettage and electrodesiccation, cryosurgery, photodynamic therapy, topical chemotherapy, topical immunotherapy (e.g., imiquimod), an intravenously administered therapeutic agent, and an orally administered therapeutic agent.

As used herein, “treat” and all its forms and tenses (including, for example, treated, and treatment) refers to therapeutic and prophylactic treatment. In certain aspects of the invention, those in need of treatment include those already with a pathological disease or condition of the invention, in which case treating refers to administering to a subject (including, for example, a human or other mammal in need of treatment) a therapeutically effective amount of a composition so that the subject has an improvement in a sign or symptom of a pathological condition of the invention. The improvement may be any observable or measurable improvement. Thus, one of the skills in the art realizes that a treatment may improve the patient's condition, but may not be a complete cure of the disease or pathological condition.

In accordance with the invention, a “therapeutically effective amount” or “effective amount” is administered to the subject. As used herein a “therapeutically effective amount” or “effective amount” is an amount sufficient to decrease, suppress, or ameliorate one or more symptoms associated with the disease or condition. In some embodiments, the dose of the one or more bioactive compounds and formulations administered ranges from about 0.1 mg to about 100 mg, from about 0.5 mg to about 50 mg, from about 1.0 mg to about 25 mg, or from about 1.0 mg to about 10 mg. In some embodiments, the dose of the one or more neurotoxins administered ranges from about 1 units to about 20 units, from about 20 units to about 40 units, from about 40 units to about 100 units, from about 100 units to about 200 units, or from about 200 units to about 400 units. Maximum cumulative dose should not exceed 400 units in 3 months.

The microchannel delivery device described herein can be used to administer therapeutic compositions one time or more than one time, for example, more than once per day, daily, weekly, monthly, or annually. The duration of treatment is not particularly limiting. The duration of administration of the therapeutic composition can vary for each individual to be treated/administered depending on the individual cases and the diseases or conditions to be treated. In some embodiments, the therapeutic composition can be administered continuously for a period of several days, weeks, months, or years of treatment or can be intermittently administered where the individual is administered the therapeutic composition for a period of time, followed by a period of time where they are not treated, and then a period of time where treatment resumes as needed to treat the disease or condition. For example, in some embodiments, the individual to be treated is administered the therapeutic composition of the invention daily, every other day, every three days, every four days, 2 days per week, 3 days per week, 4 days per week, 5 days per week or 7 days per week. In some embodiments, the individual is administered the therapeutic composition for 1 week, 2 weeks, 3 weeks, 4 weeks, 1 month, 2 months, 3 months, 4 months, 5 months, 6 months, 7 months, 8 months, 9 months, 10 months, 11 months, 1 year, or longer.

The term “subject” as used herein is not limiting and is used interchangeably with patients. In some embodiments, the term subject refers to animals, such as mammals and the like. For example, mammals contemplated include humans, primates, dogs, cats, sheep, cattle, goats, pigs, horses, chickens, mice, rats, rabbits, guinea pigs, and the like. In some embodiments, the term subject refers to pediatric subjects including infants and children in the age group of 1-12.

The term “disease or condition,” as used herein is not limiting and can include any disease or condition that can be treated with neurotoxins. In some embodiments, the disease or condition is selected from cervical dystonia, glabellar lines, forehead lines, lateral canthal lines (Crow's feet), upper limb spasticity, lower limb spasticity, blepharospasms, strabismus, sialorrhea, hemifacial spasm, axillary hyperhidrosis, chronic migraine, neurogenic detrusor overactivity, overactive bladder, urinary incontinence, Plantar Fasciitis, cerebral palsy, acquired nystagmus, anal fissures, headache, tardive dyskinesia, achalasia, eyelid convulsion, muscle spasticity, dynamic equinus foot deformity, Raynaud phenomenon, and psychological conditions such as depression.

In certain embodiments, the one or more bioactive compounds and formulations is selected from the group consisting of hyaluronic acid, botulinum toxin, platelet-rich plasma, polylactic acid, cannabinoids, vitamins, minerals, bimatoprost, minoxidil and/or other drugs or solutions. In some embodiments, the bioactive compounds are derived from Clostridium botulinum species.

In some embodiments, the one or more bioactive compounds and formulations are administered (together or separately) in combination with one or more therapies to treat the disease or conditions herein.

In some embodiments, the one or more neurotoxins is selected from FDA approved neurotoxin products including, but not limited to abobotulinumtoxinA, incobotulinumtoxinA, onabotulinumtoxinA, rimabotulinumtoxinB, prabotulinumtoxinA, daxibotulinumtoxinA, letibotulinumtoxinA, derivatives thereof and mixtures of neurotoxins. The neurotoxins can be natural or synthetically produced. Mixtures of natural and synthetically produced neurotoxins can also be used.

In some embodiments, one or more neurotoxins are administered (together or separately) in combination with one or more therapies to treat the disease or conditions herein. These combination compositions include but are not limited to, dermal fillers, such as collagen, hyaluronic acid used with neurotoxins to increase efficacy of treating glabellar lines, Crow's feet, lateral canthal lines of subjects.

In some embodiments, the combination therapy includes an antineoplastic agent. In some embodiments, the anti-neoplastic agent that can be used in methods of the present disclosure include, but are not limited to, e.g., alkylating agents and platinum compounds (e.g., cisplatin, carboplatin, oxaliplatin, mechlorethamine, cyclophosphamide, chlorambucil, ifosfamide), antimetabolic agents (e.g., purine and pyrimidine analogues, antifolates), anthracyclines (doxorubicin, daunorubicin, valrubicin, idarubicin, epirubicin), cytotoxic antibiotics (actinomycin, bleomycin, plicamycin, mitomycin), monoclonal antibodies (e.g., Alemtuzumab, Bevacizumab, Cetuximab, Gemtuzumab, Ibritumomab, Panitumumab, Rituximab, Tositumomab, and Trastuzumab), cancer antigens, checkpoint inhibitors, immune cells (activated immune cells, activated lymphocytes, engineered cells), kinase inhibitors (e.g., imatinib, erlotinib, gefitinib), plant alkaloids and terpenoids, topoisomerase inhibitors (e.g., camptothecins, irinotecan, topotecan, amsacrine, etoposide, etoposide phosphate, teniposide), vinca alkaloids (e.g., vincristine, vinblastine, vinorelbine, vindesine), taxanes (e.g., paclitaxel, taxol, docetaxel), podophyllotoxins, epipodophyllotoxins, and the like.

In some embodiments, the antineoplastic agent is selected from imiquimod, fluorouracil, vismodegib, 5-FU, sonidegib and combinations thereof. In some embodiments, these agents can be used in combination with one or more bioactive compounds and formulations to treat basal cell carcinoma.

In some embodiments, the administering comprises penetrating the microchannel delivery device into the different anatomical sites of the subject's body. These include sites in close proximity to the corrugator muscles, procerus muscles, lateral orbicularis muscles, frontalis muscles, detrusor, temporalis, occipitalis, cervical paraspinal, trapezius, biceps brachii, flexor carpi ulnaris, flexor carpi radialis, flexor digitorum sublimis (flexor digitorum superficialis), adductor pollicis, flexor digitorum profundus, flexor pollicis longus, medical head of gastrocnemius, lateral head of gastrocnemius, soleus, tibialis posterior, flexor digitorum longus and flexor hallucis longus, brachialis, brachioradialis, flexor carpi ulnaris, medial and lateral pretarsal orbicularis of upper lid, lateral pretarsal orbicularis oculi of lower lid, and intradermally to axilla.

In some embodiments, one or more supplementary administrations are administered.

In some embodiments, the bioactive compound is selected from Bevacizumab (Avastin), Ranibizumab (Lucentis), Aflibercept (Eylea) and combinations thereof. In some embodiments, these agents can be used in combination with one or more bioactive compounds and formulations to treat visual defects, degeneration, nerve repair.

In some embodiments, the bioactive compound is relenza or zanamivir in powdered or liquid form. In some embodiments, relenza can be used in combination with one or more bioactive compounds and formulations to treat influenza.

In some embodiments, the antineoplastic agent is cemiplimab-rwlc. In some embodiments, cemiplimab-rwlc can be used in combination with one or more bioactive compounds and formulations to treat cutaneous squamous cell carcinoma.

In some embodiments, the antineoplastic agent is selected from aldesleukin, cobimetinib, dabrafenib, dacarbazine, IL-2, talimogene laherparepvec, interferon alfa-2b, ipilimumab, pembrolizumab, trametinib, nivolumab, nivolumab, peg-interferon Alfa-2b, pembrolizumab, aldesleukin, dabrafenib, trametinib, vemurafenib and combinations thereof. In some embodiments, these agents can be used in combination with one or more bioactive compounds and formulations to treat melanoma.

In some embodiments, the subject administers the composition to his or her own skin.

In some embodiments, the antineoplastic agent is selected from avelumab, pembrolizumab and a combination thereof. In some embodiments, these agents can be used in combination with one or more bioactive compounds and formulations to treat Merkel cell carcinoma.

In some embodiments, the antineoplastic agent for use in combination with one or more bioactive compounds and formulations is shown below in Table 1 or 2.

TABLE 1
Exemplary antineoplastic agents (e.g., for the treatment of BCC).

LDE225B
Vismodegib
RIVEDGE
PD-L1
Patidegib
Nivolumab
Nivolumab + Ipilimumab
Imiquimod
Metvix PDT
Diclofenac
Diclofenac + Calcitriol
Calcitriol
Methyl Aminolevulinate
Fractionated 5-aminolevulinic acid hydrochloride
PEP005
SUBA-Itraconazole
methyl-aminolevulinate
Remetinostat
verteporfin PDT
Sonidegib
Itraconazole
vismodegib
Picato
Resiquimod
API 31510
Aminolevulinic acid
arsenic trioxide
Patidegib
REGN2810
Buparlisib
Oshadi D & Oshadi R
Celecoxib
Tazarotene
Sinecatechins 10%
aminolevulinic acid hydrochloride
Vismodegib
FOLFOX
FOLFIRI
Bevacizumab
Tazarotene
Hexaminolevulinate
Aminolevulinic Acid Nano Emulsion
Methyl Aminolevulinate
liposomal T4N5 lotion
Carboplatin
Cyclophosphamide
Etoposide
Methotrexate
Vincristine Sulfate
Acetylcysteine
Bevacizumab
Eflornithine
Celecoxib
erlotinib hydrochloride
Poly-ICLC
aminolevulinic acid
eflomithine
triamcinolone
18F-fludeoxyglucose (18F-FDG)
18F-FPPRGD2
Fluconazole
cevimeline hydrochloride
megestrol acetate
Amifostine
Carboplatin
Etoposide
Ifosfamide
Hypericum perforatum
Docetaxel
Nicotinamide
doxepin hydrochloride
capecitabine
oxaliplatin
DetoxPC
Capecitabine
Carboplatin
epirubicin hydrochloride
cisplatin
paclitaxel
Tretinoin
doxorubicin hydrochloride
gemcitabine hydrochloride
indinavir sulfate
ritonavir
5-fluorouracil

TABLE 2
Exemplary antineoplastic agents (e.g., for
the treatment of actinic keratosis).

Imiquimod
VDA-1102
fluorouracil
Cetaphil
SOR007 (Uncoated Nanoparticulate Paclitaxel)
Aminolevulinic Acid
PEP 005 (ingenol mebutate)
ingenol disoxate
Ingenol Mebutate
Aminolevulinic Acid (ALA)
Biafine
Polysporin
5-FU
A-101 Solution (High-Concentration Hydrogen Peroxide)
lidamycin phosphate and benzoyl peroxide 1.2%/3.75% combination
Eflornithine
Triamcinolone
Polyphenon E
BLU-U
ACT01
Levulan ®
Kerastick ®
perillyl alcohol
aminolevulinic acid
liposomal T4N5
Aminolevulinic Acid
GDC 695
Diclofenac Sodium
KX2-391
ingenol disoxate
ingenol disoxate
Celecoxib
Bupivacaine + Clonidine
Secukinumab
Pimecrolimus

A microchannel delivery device is used to deliver the therapeutic composition. In some embodiments, the microchannel delivery device is shown in FIGS. 1-15. In some embodiments, the microneedle drug delivery device is as described in U.S. Pat. No. 10,980,865 and Korean Patent No. 10-1582822, which are incorporated by reference herein in their entirety.

In some embodiments, the microchannel delivery device comprises

i) a plurality of modular or replaceable chambers, wherein the chambers can hold the bioactive compounds or formulations;

ii) a plurality of microneedles, wherein the microneedles are hollow or non-hollow, wherein one or multiple grooves are inset along an outer wall of the microneedles; and

iii) a reservoir that holds the composition to be delivered, wherein the reservoir is attached to or contains a means to encourage flow of the bioactive composition contained in the reservoir into the skin.

iv) a spring system that enables tap and deliver mechanism of administering composition into the skin

In some embodiments, the composition is administered by the microchannel delivery device through a single motion of penetrating the microchannel delivery device into the skin of the subject through a plunger mechanism. In some embodiments, the composition is delivered into the skin by passing through the one or multiple grooves along the outer wall of the microneedle. In some embodiments, the microneedles are non-hollow.

In some embodiments, the composition is administered by the microchannel delivery device with a repeated motion of penetrating the microchannel delivery device into the skin of the subject. In some embodiments, the composition is delivered into the skin by passing through the one or multiple grooves along the outer wall of the microneedle. In some embodiments, the microneedles are non-hollow.

In some embodiments, the chamber contains a pin that punctures the other chamber to allow flow of bioactive formulation from one chamber to another chamber. In some embodiments, the pin is pushed by an external pusher as described in FIG. 1-15.

In some embodiments, the microchannel delivery device is modular as described in FIG. 12. In some embodiments, each chamber of the device can be removed and added to the device through a push pin, mechanical or magnetic fittings.

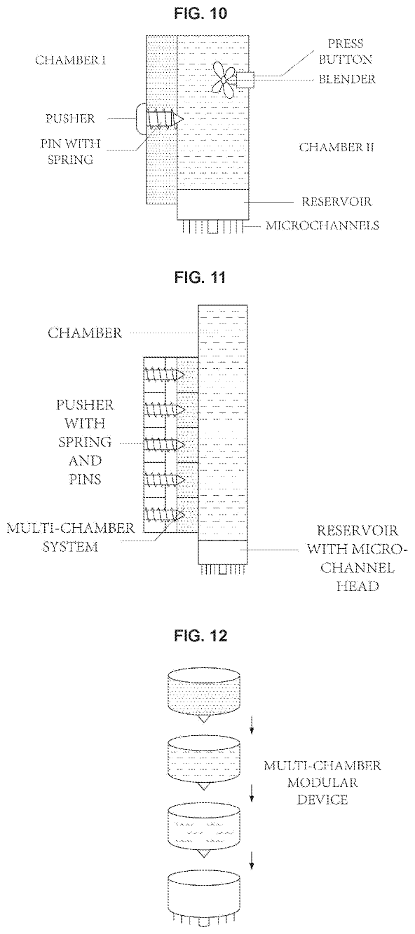

In some embodiments, the chamber contains a blender that facilitates the mixing of the bioactive compounds as described in FIG. 10.

In some embodiments, the lining between the chambers are made of plastic films with low puncture resistance. In some embodiments, the lining between the chambers are made of deformable, preferably elastic, material.

In some embodiments, the means to encourage flow of the composition contained in the reservoir into the skin is selected from the group consisting of a plunger, pump and suction mechanism. In some embodiments, the means to encourage flow of the composition contained in the reservoir into the skin is a mechanical spring loaded pump system.

In some embodiments, the chambers can hold a bioactive formulation in a powder form or in an aqueous solution.

In some embodiments, the means to encourage the flow of the composition contained in the reservoir into the skin is selected from the group consisting of a plunger, pump and suction mechanism. In some embodiments, the means to encourage the flow of the composition contained in the reservoir into the skin is a mechanical spring loaded pump system.

In some embodiments, the microneedle drug delivery device facilitates auto-reconstitution of composition within the chambers in the reservoir. In some embodiments, the auto-reconstitution is facilitated by the blender as described in FIG. 10.

In some embodiments, the microneedle delivery device is connected to an external pressure-based (pneumatic) pump system to facilitate the movement of bioactive formulation from one chamber to another, or from the reservoir to the microneedle head, and during administration.

In some embodiments, the microneedle delivery device is connected to an external electrically-powered pump system to facilitate the movement of bioactive formulation from one chamber to another, or from the reservoir to the microneedle head, and during administration.

In some embodiments, the microneedle delivery device consists of negative pressure-induced chambers to automatically release compounds when exposed to external atmospheric pressure, to facilitate the movement of bioactive formulation from one chamber to another, or from the reservoir to the microneedle head, and during administration.

In some embodiments, the microneedles have a single groove inset along the outer wall of the microneedle, wherein the single groove has a screw thread shape going clockwise or counterclockwise around the microneedle.

In some embodiments, the microneedles are from 0.1 mm to about 25 mm in length and from 0.01 mm to about 0.05 mm in diameter.

In some embodiments, the length of the microneedles can be changed

In some embodiments, the microneedles are made from a substance comprising gold.

In some embodiments, the plurality of microneedles comprises an array of microneedles in the shape of a circle.

In some embodiments, the microneedles are made of 24-carat gold plated stainless steel and comprise an array of about 10 to about 50 microneedles. In some embodiments, the array comprises 20 microneedles.

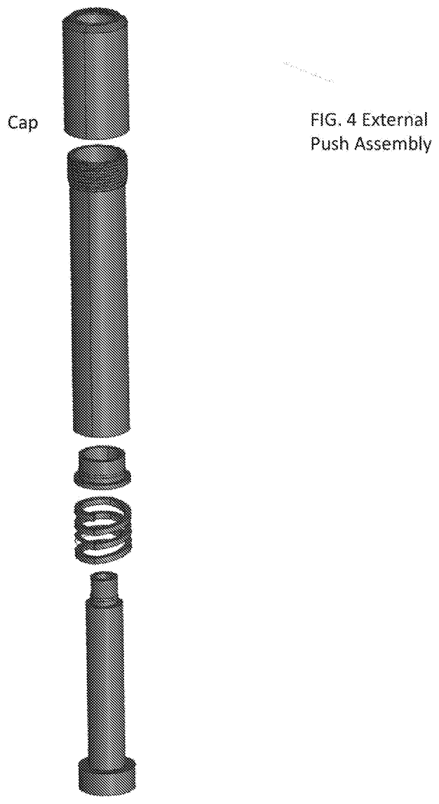

In some embodiments, the microchannel delivery device is pressed once against the subject's skin and the distal end of the external push assembly (Refer FIGS. 4-6) is pushed to deliver the composition to the area of the skin to be treated.

In some embodiments, the microchannel delivery device comprises a single or an array of microneedles. In some embodiments, the microneedles will have one or multiple grooves inset along its outer wall. This structural feature of the dermal delivery device allows liquids stored in a reservoir at the base of each needle to travel along the needle shaft into the tissue.

In some embodiments, the microneedle array comprises from about 1 to about 500 microneedles, which will be anywhere from about 0.1 to about 25 mm in length and from 0.01 to about 0.5 mm in diameter, and be composed of any metal, metal alloy, metalloid, polymer, or combination thereof, such as gold, steel, silicon, PVP (polyvinylpyrrolidone), etc. The microneedles will each have one or more recesses running a certain depth into the outer wall to allow for flow of the substance to be delivered down the microneedle and into the dermis; these recesses can be in a plurality of shapes, including but not limited to: straight line, cross shape (+), flat shape (−), or screw thread shape going clockwise or counterclockwise. The array will be in any shape or combination of shapes, continuous, or discontinuous. The list of possible shapes includes, but is not limited to, circles, triangles, rectangles, squares, rhomboids, trapezoids, and any other regular or irregular polygons. The array can be attached to a reservoir to hold the substances to be delivered, and this reservoir will be any volume (0.25 mL to 5 mL), shape, color, or material (glass, metal, alloy, or polymer), as determined necessary. This reservoir will itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

The microchannel delivery device is capable of delivering compositions directly to the epidermal, dermal, subcuticular and intramuscular layers of the skin. Therefore, it should be understood that further embodiments developed for use with non-hollow or hollow microneedle systems of delivery by those skilled in the art fall within the spirit and scope of this disclosure.

In another aspect, a microchannel delivery device for use in the methods described herein is a device such as described in the U.S. Pat. No. 8,257,324, which is hereby incorporated by reference. Briefly, the devices include a substrate to which a plurality of hollow microneedles are attached or integrated, and at least one reservoir, containing a bioactive formulation, selectably in communication with the microneedles, wherein the volume or amount of composition to be delivered can be selectively altered. The reservoir can be, for example, formed of a deformable, preferably elastic, material. The device typically includes a means, such as a plunger, for compressing the reservoir to drive the bioactive formulation from the reservoir through the microneedles, A reservoir, can be, for example, a syringe or pump connected to the substrate. A device, in some instances, comprises: a plurality of hollow microneedles (each having a base end and a tip), with at least one hollow pathway disposed at or between the base end and the tip, wherein the microneedles comprise a metal; a substrate to which the base ends of the microneedles are attached or integrated; at least one reservoir in which the material is disposed and which is in connection with the base end of at least one of the microneedles, either integrally or separably; a sealing mechanism interposed between the at least one reservoir and the substrate, wherein the sealing mechanism comprises a fracturable barrier; and a device that expels the material in the reservoir into the base end of at least one of the microneedles and into the skin. The reservoir comprises a syringe secured to the substrate, and the device that expels the material comprises a plunger connected to a top surface of the reservoir. The substrate may be adapted to removably connect to a standard or Luer-lock syringe. In one instance, the device may further include a spring engaged with the plunger. In another instance, the device may further include an attachment mechanism that secures the syringe to the device. In another instance, the device may further include a sealing mechanism that is secured to the tips of the microneedles. In another instance, the device may further include means for providing feedback to indicate that delivery of the material from the reservoir has been initiated or completed. An osmotic pump may be included to expel the material from the reservoir. A plurality of microneedles may be disposed of at an angle other than perpendicular to the substrate. In certain instances, at least one reservoir comprises multiple reservoirs that can be connected to or are in communication with each other. The multiple reservoirs may comprise a first reservoir and a second reservoir, wherein the first reservoir contains a solid formulation and the second reservoir contains a liquid carrier for the solid formulation. A fracturable barrier for use in the devices can be, for example, a thin foil, a polymer, a laminate film, or a biodegradable polymer. The device may further comprise, in some instances, means for providing feedback to indicate that the microneedles have penetrated the skin.

In some embodiments, the device can include, in some instances, a single or plurality of solid, screw-type microneedles, of single or varied length. Typically the needles attach to a substrate or are embedded within the substrate. The substrate can be made of any metal, metal alloy, ceramics, organics, metalloid, polymer, or combination thereof, including composites, such as gold, steel, silicon, PVP (polyvinylpyrrolidone) etc. The screw-shape dimensions may be variable. For example, in one embodiment the screw-shape may be a tight coiled screw shape, whereas in another embodiment the screw-shape might be a loose coiled screw shape whereby the screw threads in one embodiment lie closely together along the outer edge of the needle and, in another embodiment, the screw threads lie far from each other along the outer edge of the needle.

In one embodiment a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solutions from the reservoir through to the dermis.

A device described herein may contain, in certain instances, about twenty screw thread design surgical grade microneedles. Each microneedle has a diameter that is thinner than a human hair and may be used for direct dermal application. In one instance, a microneedle has a diameter of less than about 0.18 mm. In another instance, a microneedle has a diameter of about 0.15 mm, about 0.14 mm, about 0.13 mm, about 0.12 mm, about 0.11 mm, or about 0.10 mm. Each microneedle may be plated with 24 carat gold. The device allows for targeted and uniform delivery of a composition comprising one or more neurotoxins into the skin in a process that is painless compared to injectables. Administration can result in easy and precise delivery of a composition comprising one or more neurotoxins with generally no bruising, pain, swelling and bleeding. Delivery of one or more neurotoxins may include sensitive areas and areas difficult to treat with traditional methods, such as around the eyes and mouth.

The device may include means, manual or mechanical, for compressing the reservoir, creating a vacuum, or otherwise using gravity or pressure to drive the one or more neurotoxins from the reservoir through the microneedles or down along the sides of the microneedle. The means can include a plunger, pump or suction mechanism. In another embodiment, the reservoir further includes a means for controlling rate and precise quantity of drug delivered by utilizing a semi-permeable membrane, to regulate the rate or extent of drug which flows along the shaft of the microneedles. The microchannel delivery device enhances transportation of drugs across or into the tissue at a useful rate. For example, the microchannel delivery device must be capable of delivering drugs at a rate sufficient to be therapeutically useful. The rate of delivery of the drug composition can be controlled by altering one or more of several design variables. For example, the amount of material flowing through the needles can be controlled by manipulating the effective hydrodynamic conductivity (the volumetric through-capacity) of a single device array, for example, by using more or fewer microneedles, by increasing or decreasing the number or diameter of the bores in the microneedles, or by filling at least some of the microneedle bores with a diffusion-limiting material. It can be preferred, however, to simplify the manufacturing process by limiting the needle design to two or three “sizes” of microneedle arrays to accommodate, for example small, medium, and large volumetric flows, for which the delivery rate is controlled by other means.

Other means for controlling the rate of delivery include varying the driving force applied to the drug composition in the reservoir. For example, in passive diffusion systems, the concentration of drugs in the reservoir can be increased to increase the rate of mass transfer. In active systems, for example, the pressure applied to the reservoir can be varied, such as by varying the spring constant or number of springs or elastic bands. In either active or passive systems, the barrier material can be selected to provide a particular rate of diffusion for the drug molecules being delivered through the barrier at the needle inlet.

The array may be in any shape or combination of shapes, continuous, or discontinuous. The list of possible shapes includes, but is not limited to, circles, triangles, rectangles, squares, rhomboids, trapezoids, and any other regular or irregular polygons.

The array may be attached to a reservoir to hold the substances to be delivered, and this reservoir may be any volume (about 0.25 mL to about 5 mL), shape, color, or material (glass, metal, alloy, or polymer), as determined necessary.

This reservoir can itself be attached to or contain a means to encourage flow of the drug solutions contained in the reservoir into the skin. Two non-limiting examples of such means are 1) a plate and spring that allows the contained solutions to flow only when the device is tapped into the skin, and 2) a syringe that contains the drug solutions to be delivered and includes a plunger that can be depressed to mechanically drive the solution into the skin.

In some embodiments, the device can include a single or plurality of solid, screw-type microneedles, of single or varied lengths housed in a plastic or polymer composite head which embodies a corrugated rubber connector. In some embodiments, the needles attach to a substrate or are embedded within the substrate. The substrate can be made of any metal, metal alloy, ceramics, organics, metalloid, polymer, or combination thereof, including composites, such as gold, steel, silicon, PVP (polyvinylpyrrolidone) etc. The screw-shape dimensions may be variable. For example, in one embodiment the screw-shape may be a tight coiled screw shape, whereas in another embodiment the screw-shape might be a loose coiled screw shape. The corrugated rubber connector is a unique advantage conferring feature which bestows the microneedle head with a universally adoptable feature for interfacing the micro needle cartridges with multiple glass and or plastic vials, reservoirs and containers as well as electronic appendages for an altogether enhanced adjunct liquid handling, security and surveillance utility.

In one embodiment a reservoir would attach to the substrate to allow drug solution to flow down the side of the microneedles. In one embodiment the reservoir is a solid canister of differing sizes depending on the desired volume or amount of drug to be delivered. The reservoir contains the drug to be delivered. In another embodiment, the reservoir can be supported by a mechanical (spring loaded or electrified machine-driven) pump system to deliver the drug solution. In another embodiment, the reservoir is composed of a rubber, elastic, or otherwise deformable and flexible material to allow manual squeezing to deliver the drug solution. In another embodiment the device includes hollow needles or needles with alternative ridges and shapes to more efficiently drive solutions from the reservoir through to the dermis.

The amount of the therapeutic agents of the invention which will be effective in promoting a therapeutic effect can be determined by standard clinical techniques. The precise dose to be employed in the formulation will also depend on the judgment of the practitioner and each subject's circumstances. Effective doses can be extrapolated from dose-response curves derived from in vitro or animal model test systems.

One skilled in the art can readily determine an appropriate dosage regimen for administering neurotoxins of the invention to a given subject. For example, the compound(s) or composition(s) can be administered to the subject in one administration or multiple administrations. Where a dosage regimen comprises multiple administrations, it is understood that the effective amount of the compound(s) or composition(s) administered to the subject can comprise the total amount of the compound(s) or composition(s) administered over the entire dosage regimen. The exact amount will depend on the purpose of the treatment, the subject to be treated, and will be ascertainable by a person skilled in the art using known methods and techniques for determining effective doses. In some embodiments, the amount of the therapeutic agent that can be administered includes about 4 units/0.1 mL to about 20 units total for treatment of glabellar lines. In some embodiments, the amount of the one or more neurotoxins that can be administered includes about 1 unit/0.1 mL to about 100 units total for treatment of overactive bladder to about 10 mg/kg.

In some embodiments, the neurotoxins are formulated in accordance with routine procedures as a pharmaceutical composition adapted for intramuscular, subcutaneous or parenteral administration to human beings. In some embodiments, compositions for administration are solutions in sterile isotonic aqueous buffers. Where necessary, the composition can also include a solubilizing agent. Generally, the ingredients are supplied either separately or mixed together in unit dosage form, for example, as a dry lyophilized powder or water free concentrate in a hermetically sealed container such as an ampoule indicating the quantity of active agent. In some embodiments, an ampoule of sterile water for injection or saline can be provided so that the ingredients can be mixed prior to administration.

In certain embodiments, the compositions are pharmaceutical compositions. In some embodiments, formulations are prepared for storage and use by combining the active agents with a pharmaceutically acceptable vehicle (e.g. carrier, excipient) (Remington, The Science and Practice of Pharmacy 20th Edition Mack Publishing, 2000). In some embodiments, pharmaceutical compositions of the present invention are characterized as being at least sterile and pyrogen-free. As used herein, “pharmaceutical formulations” include formulations for human and veterinary use. Pharmaceutical compositions of the invention can be packaged for use in liquid form, or can be lyophilized.

Suitable pharmaceutically acceptable vehicles include, but are not limited to, sterile vehicles, nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g. less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG).

For a broad overview of controlled delivery systems, see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nanospheres, and nanoparticles. Microcapsules can contain therapeutically active agents as a central core. In microspheres the therapeutic can be dispersed throughout the particle. Particles, microspheres, and microcapsules smaller than about 1 μm are generally referred to as nanoparticles, nanospheres, and nanocapsules, respectively. Microparticles are typically around 100 μm in diameter. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker Inc. New York, N.Y., pp. 315-339, (1992).

In some embodiments, polymers can be used for controlled release of compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). In yet another aspect, liposomes can be used for controlled release as well as drug targeting of the lipid-encapsulated drug (Betageri et al., Liposome Drug Delivery Systems, Technomic Publishing Co., Inc., Lancaster, Pa. (1993)).

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

1. A method for treating a condition or disease in a subject, comprising administering to the subject's skin a composition comprising an effective amount of one or more bioactive formulations, including neurotoxins, wherein the composition is administered with a single-chamber or multi-chamber microchannel delivery device.

2. The method of claim 1, wherein the microchannel delivery device comprises
i) a plurality of modular or replaceable chambers, wherein the chambers can hold the bioactive compounds or formulations;
ii) a plurality of microneedles, wherein the microneedles are hollow or non-hollow, wherein one or multiple grooves are inset along an outer wall of the microneedles;
iii) a reservoir that holds the composition to be delivered, wherein the reservoir is attached to or contains a means to encourage flow of the bioactive composition contained in the reservoir into the skin; and
iv) a spring system that enables tap and deliver mechanism of administering composition into the skin.
wherein the administering comprises pressing the distal end of an external push assembly to enable a repeated motion of penetrating the microchannel delivery device into the skin of the subject,
wherein the composition is delivered into the skin by passing through the one or multiple grooves along the outer wall of the microneedle.

3. The method of claim 2, wherein the microneedles are hollow or non-hollow.

4. The method of claim 1, wherein the means to encourage flow of the composition contained in the reservoir into the skin is selected from the group consisting of a plunger, pump and suction mechanism.

5. The method of claim 4, wherein the means to encourage flow of the composition contained in the reservoir into the skin is a mechanical spring loaded pump system.

6. The method of claim 1, wherein the microneedles have a single groove inset along the outer wall of the microneedle, wherein the single groove has a screw thread shape going clockwise or counterclockwise around the microneedle.

7. The method of claim 1, wherein the microneedles are from 0.1 mm to about 25 mm in length and from 0.01 mm to about 0.05 mm in diameter.

8. The method of claim 1, wherein the microneedles are made from a substance comprising gold.

9. The method of claim 2, wherein the plurality of microneedles comprises an array of microneedles in the shape of a circle.

10. The method of claim 1, wherein the microneedles are made of 24-karat gold plated stainless steel and comprise an array of 20 microneedles.

11. The method of claim 1, wherein the one or more bioactive formulations are selected from the group consisting of hyaluronic acid, botulinum toxin, platelet-rich plasma, polylactic acid, cannabinoids, vitamins, minerals, bimatoprost, and minoxidil.

12. The method of claim 1, wherein the one or more bioactive formulations are administered with an effective amount of an antineoplastic agent.

13. The method of claim 12, wherein the antineoplastic agent is selected from the group consisting of alkylating agents, platinum compounds (e.g., cisplatin, carboplatin, oxaliplatin, mechlorethamine, cyclophosphamide, chlorambucil, ifosfamide), antimetabolic agents (e.g., purine and pyrimidine analogues, antifolates), anthracyclines (doxorubicin, daunorubicin, valrubicin, idarubicin, epirubicin), cytotoxic antibiotics (actinomycin, bleomycin, plicamycin, mitomycin), monoclonal antibodies (e.g., Alemtuzumab, Bevacizumab, Cetuximab, Gemtuzumab, Ibritumomab, Panitumumab, Rituximab, Tositumomab, and Trastuzumab), kinase inhibitors (e.g., imatinib, erlotinib, gefitinib), plant alkaloids and terpenoids, topoisomerase inhibitors (e.g., camptothecins, irinotecan, topotecan, amsacrine, etoposide, etoposide phosphate, teniposide), vinca alkaloids (e.g., vincristine, vinblastine, vinorelbine, vindesine), taxanes (e.g., paclitaxel, taxol, docetaxel), podophyllotoxins, epipodophyllotoxins and combinations thereof.

14. (canceled)

15. The method of claim 1, wherein the disease or condition is selected from the group consisting of anorexia, emesis, sunburn, photodamaged skin, acne, psoriasis, atopic dermatitis, pain, inflammation, multiple sclerosis, neurodegenerative disorders (e.g. Parkinson's disease, Huntington's disease, Tourette's syndrome, Alzheimer's disease), epilepsy, glaucoma, osteoporosis, schizophrenia, cardiovascular disorders, cancer, actinic keratosis, obesity, and metabolic syndrome-related disorders.

16. The method of claim 1, wherein the disease or condition is selected from cervical dystonia, glabellar lines, forehead lines, lateral canthal lines (Crow's feet), upper limb spasticity, lower limb spasticity, blepharospasms, strabismus, sialorrhea, hemifacial spasm, axillary hyperhidrosis, chronic migraine, neurogenic detrusor overactivity, overactive bladder, urinary incontinence, Plantar Fasciitis, cerebral palsy, acquired nystagmus, anal fissures, headache, tardive dyskinesia, achalasia, eyelid convulsion, muscle spasticity, dynamic equinus foot deformity, Raynaud phenomenon, and psychological conditions such as depression.

17. The method of claim 1, further comprising administering to the subject one or more additional therapies.

18. The method of claim 16, wherein the additional therapy includes radiation, surgery, chemotherapy, simple excision, Mohs micrographic surgery, Curettage and electrodesiccation, cryosurgery, photodynamic therapy, topical chemotherapy, topical immunotherapy, intravenously administered therapeutic agent, and orally administered therapeutic agent.

19. A single-chamber or multi chamber microneedle drug delivery device comprising a composition comprising an effective amount of one or more bioactive formulations.

20. The single-chamber or multi-chamber microneedle drug delivery device of claim 19, wherein the one or more bioactive formulations are in lyophilized or powder form.

21. The single-chamber or multi-chamber microneedle drug delivery device of claim 19, wherein the one of more bioactive formulations are auto-reconstituted.
